(19) United States
(12) Patent Application Publication
(10) Pub. No.: US 2004/0096873 A1
(43) Pub. Date: May 20, 2004

(54) Genotype-dependent fibrinolysis changes with exercise training

(76) Inventors: James M. Hagberg, Columbia, MD (US); Onanong Kulaputana, Bangkok (TH); Robert E. Ferrell, Pittsburgh, PA (US)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/612,894

(22) Filed: Jul. 7, 2003

Related U.S. Application Data
(60) Provisional application No. 60/393,970, filed on Jul. 5, 2002.

(51) Int. Cl. C12Q 1/68

(52) U.S. Cl. 435/6

(57) ABSTRACT

Methods of preventing or alleviating cardiovascular disease, as well as increasing fibrinolysis levels in a subject, through the identification of subjects with an allele and/or genotype at a gene locus that positively correlates with improved success in increasing fibrinolysis, as compared to other alleles and/or genotypes at the same gene locus, and through engagement of these subjects in exercise training for a period of time sufficient to increase fibrinolysis.

This application claims priority under 35 U.S.C. 119(e) based upon U.S. Provisional Application No. 60/393,970, filed on Jul. 5, 2002.

### FIELD OF THE INVENTION

The present invention relates to identifying one or more genetic markers which correlate with improved success in increasing fibrinolysis levels in sedentary individuals with exercise training.

### BACKGROUND OF THE INVENTION

Fibrinolysis is the proteolytic cleavage of fibrin by plasmin to produce fibrinopeptides. It is also the process by which a fibrin clot is removed from the site of vascular injury during the healing process. During this process, damaged endothelial cells release tissue plasminogen activator, which converts plasminogen into plasmin. The plasmin, in turn, cleaves fibrin into circulating fibrin degradation products.

Prospective epidemiological investigations suggest that impaired fibrinolysis is an independent risk factor for cardiovascular disease. It has been demonstrated in many publications (including Meade T W, Ruddock V, Stirling Y, Chakrabarti R, Miller G J. Fibrinolytic activity, clotting factors, and long-term incidence of ischaemic heart disease in the Northwick Park Heart Study. Lancet 1993; 342:1076-1079; Hamsten A, de Faire U, Walldius G, Dahlen G, Szamosi A, Landou C, Blomback M, Wiman B. Plasminogen activator inhibitor in plasma: risk factor for recurrent myocardial infarction. Lancet 1987; 2:3-9; Cortellaro M, Cofrancesco E, Boschetti C, Mussoni L, Donati M B, Cardillo M, Catalano M, Gabrielli L, Lombardi B, Specchia G. Increased fibrin turnover and high PAI-1 activity as predictors of ischemic events in atherosclerotic patients. A case-control study. The PLAT Group. Arterioscler Thromb 1993; 13:1412-1417; Juhan-Vague I, Pyke S D, Alessi M C, Jespersen J, Haverkate F, Thompson S G. Fibrinolytic factors and the risk of myocardial infarction or sudden death in patients with angina pectoris. ECAT Study Group. European Concerted Action on Thrombosis and Disabilities. Circulation 1996; 94:2057-2063; Ridker P M, Vaughan D E, Stampfer M J, Manson J E, Hennekens C H. Endogenous tissue-type plasminogen activator and risk of myocardial infarction. Lancet 1993; 341:1165-1168; and Ridker P M, Hennekens C H, Stampfer M J, Manson J E, Vaughan D E. Prospective study of endogenous tissue plasminogern activator and risk of stroke. Lancet 1994; 343:940-943, all of which are herein incorporated by reference in their entirety) that increased levels of fibrinolysis is associated with decreased cardiovascular disease.

However, endurance exercise training has been shown to have an inconsistent effect on fibrinolysis, possibly due to inadequate study design and lack of control of critical variables. It is clear from these studies that some individuals suffering from cardiovascular disease can alleviate symptoms or otherwise improve their conditions through exercise. Unfortunately, these studies also reveal that some individuals, no matter how rigorously they exercise, are unable to improve their conditions, while others benefit to a much greater extent than predicted. These results underscore the fact that many factors contribute to an individual's well-being. Such factors include, for example, behaviors such as diet and exercise, genetic makeup, and environment. While behavior and environment can be controlled, altered or regulated, an individual's genetic makeup is essentially predetermined and set at birth. The present inventors hypothesized that upon identifying the genetic makeup of a sedentary population and observing that some individuals of the population increase their levels of fibrinolysis from a change of behavior (such as exercise training) to a much greater or lesser extent than expected, a correlation could be made between the presence or absence of certain genetic markers and success in increasing fibrinolysis levels with the change of behavior.

### SUMMARY OF THE INVENTION

The present inventors have discovered a number of genetic markers which positively correlate with improved success in increasing fibrinolysis levels in sedentary individuals with exercise training, as compared with other genetic makeup at the same gene loci. Therefore, the present invention is directed to a method of increasing fibrinolysis levels comprising:

identifying a subject having an allele and/or genotype at a particular gene locus which positively correlates with improved success in increasing fibrinolysis levels in individuals, as compared with other alleles and/or genotypes at the same gene locus, and

engaging the subject in exercise training for a period of time sufficient to increase fibrinolysis levels in the subject.

The present inventors also discovered that improving the levels of fibrinolysis in subjects also prevented the development of cardiovascular disease or alleviated symptoms of cardiovascular disease already in existence. Therefore, the present invention is also directed to methods of preventing cardiovascular disease or ameliorating the symptoms of already existing cardiovascular disease, the methods comprising: identifying a subject having an allele and/or genotype at a particular gene locus which positively correlates with improved success in increasing fibrinolysis levels in individuals, as compared with other alleles and/or genotypes at the same gene locus, and

engaging the subject in exercise training for a period of time sufficient to prevent cardiovascular disease or ameliorate the symptoms of already existing cardiovascular disease in the subject.

### BRIEF DESCRIPTION OF THE FIGURES

FIG. 1 describes changes in the t-PA activity with exercise training among the three genotypes.

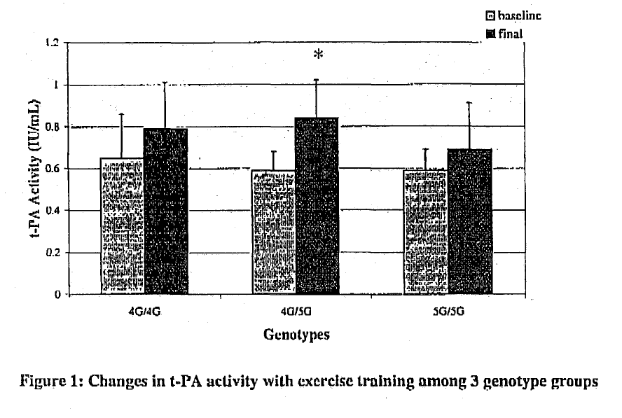

FIG. 2 describes changes in t-PA antigen levels with exercise training among the three genotypes.

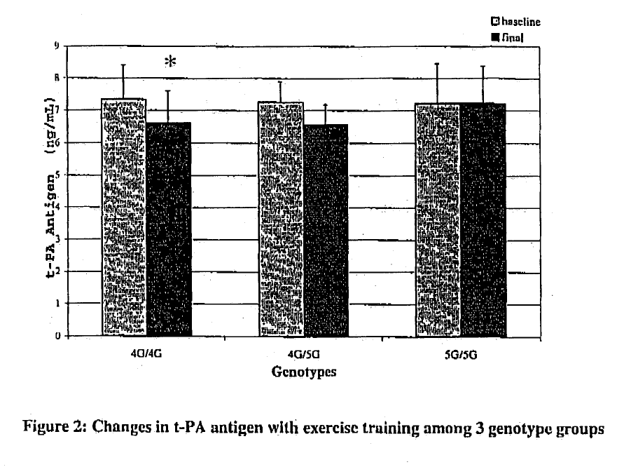

FIG. 3 illustrates a scatter plot of baseline and final t-PA activity among the three genotypes.

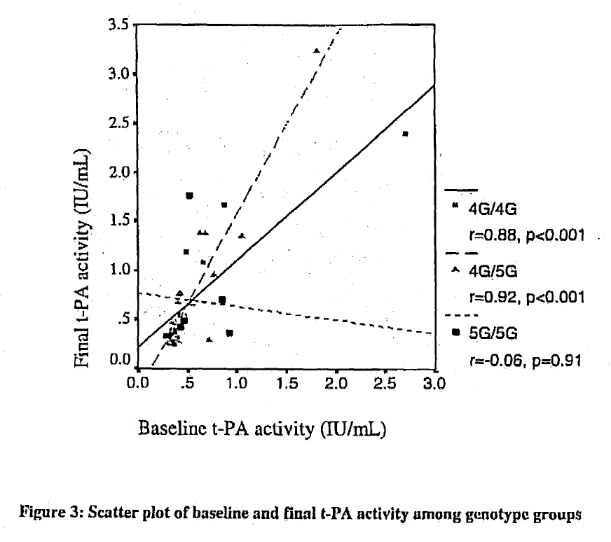

### DETAILED DESCRIPTION OF THE INVENTION

The inventors have found that a number of genetic markers positively correlate with improved success in increasing fibrinolysis levels in sedentary individuals with exercise training, as compared with other genetic makeups at the same gene loci. The inventors have investigated the plasminogen activator inhibitor-I (PAI-1) gene promoter site, in particular genotypes 4G/5G, 4G/4G, and 5G/5G.

PAI-1 acts to inhibit the fibrinolysis process by inhibiting the actions of t-PA. t-PA is a tissue plasminigen activator and its presence stimulates fibrinolysis. By inhibiting t-PA, PAI-1, therefore, also inhibits fibrinolysis.

The term “t-PA antigen” is used herein to refer to the t-PA composition that stimulates fibrinolysis.

The term “t-PA activity” is a description of the rate of chemical and enzymatic reactions caused by the presence of t-PA antigen with and without the presence of PAI-1.

The term “sedentary” is herein defined as not having participated in regular aerobic activity for at least 6 months, that is, on average the regular aerobic activity should not have exceeded 20 minutes, 2 times per week.

The term “healthy” is herein defined as a positive description of the physical health of a subject or the soundness of the overall condition of the subject.

The term “middle-aged” is intended to describe an age span of 50-70 years of age in a subject.

The term “single course of exercise,” as used throughout this application, means a cardiovascular exercise session of any type which is conducted during one day. An exercise session may comprise an aerobics class, treadmill training, step machine, or any other suitable cardiovascular exercise regimen. For most cases, exercise may be completed in, for example, 15 minutes to 3 hours, with optional brief rest periods of 3-15 minutes, however this amount would vary depending on the health and endurance of the subject.

The term “extensive exercise” means about 10 single courses of exercise or more, preferably at least 15, at least 20, or at least 25 single courses of exercise, over a defined period of time (“the exercise period”). The exercise period in the case of an extensive exercise protocol may be from about 30-400 days, preferably from about 50-350 days or 70-300 days.

The term “moderate exercise” means about 5-9 single courses of exercise, preferably about 6-8, or 7 single courses of exercise, over the exercise period. The exercise period in the case of a moderate exercise protocol may be from about 5-50 days, preferably from about 5-30 days, 5-20 days, or 5-15 days.

The term “limited exercise” means about 5 single courses of exercise or less, preferably at most 3, or 1 single course of exercise, over the exercise period. The exercise period in the case of a limited exercise protocol may be about 12 days or less, preferably at most 10, at most 7, or at most 5 days. It is most preferred that the limited exercise period be at most 3 days.

A period of time sufficient to increase fibrinolysis, prevent cardiovascular disease, or prevent cardiovascular disease when engaged in extensive, moderate, or limited exercise is about 70-400 days, preferably from about 50-350 days, more preferably from about 70-300 days, and even more preferably 100-180 days.

The time between exercise periods depends on whether the exercise period is an extensive, limited or moderate exercise period. In the case of extensive exercise periods, the time between exercise periods may be from about 10-120 days or more. In the case of limited exercise periods, the time between exercise periods may be from 4-60 days or more. In the case of moderate exercise periods, the time between exercise periods may be from 6-90 days or more. The term “between exercise periods” means that time during which the subject is not in an extensive, limited or moderate exercise program.

It has been determined that the PAI-1 gene promoter 4G/5G polymorphism affects circulating PAI-1 levels. It has also been determined that the PAI-1 polymorphism and initial PAI-1 levels play roles in exercise training-induced fibrinolysis changes. Therefore, it is possible to increase fibrinolysis in a subject by adapting a subject's exercise regimen to correspond to his or her genotype. Said subject may be of any age, preferably middle-aged and older, and most preferably middle-aged. Such a process would comprise identifying the individual's genotype and then determining if the individual possesses at least one 4G polymorphism. If so, the individual will respond to extensive, moderate, or limited exercise training for a time sufficient to increase fibrinolysis, thereby increasing his or her t-PA activity levels and decrease the inhibition of fibrinolysis by PAI-1.

Likewise, it is possible to alleviate or prevent cardiovascular disease by examining the genotype of an individual and assigning a suitable exercise regimen based upon the individual's genotype. As noted earlier, cardiovascular disease has been linked with inhibition of fibrinolysis. Therefore, an increase in fibrinolytic activity would at least alleviate, or even prevent development of, cardiovascular disease. A process for preventing or alleviating cardiovascular disease would comprise examining the genotype of a subject to determine whether he or she possessed at least one 4G polymorphism. Said subject may be of any age, preferably middle-aged and older, and most preferably middle-aged. If so, then the process would comprise placing that subject on a regimen of extensive, moderate, or limited exercise for a time sufficient to alleviate or prevent cardiovascular disease. A time sufficient for preventing cardiovascular disease is the same, but it is preferred that the subject remain on the exercise regimen, not only for apparent physical health reasons, but also to maintain the benefit of preventing the cardiovascular disease.

### EXAMPLES

These determinations were confirmed through testing of sedentary healthy volunteers aged 50-70 years. In the studies, the subjects completed a 6-week dietary program before baseline testing. The baseline tests of 48 subjects included fibrinolytic enzymes (tissue plasminogen activator (t-PA) and PAI-1 activities, and t-PA antigen), lipid-lipoprotein profiles, body composition, and maximal oxygen consumption (VO2 max). Genotyping was performed using PCR/RFLP techniques. Baseline tests were repeated in 34 subjects after 6-month exercise intervention.

### Example 1

### Subject Selection and Screening

Subjects were selected from sedentary and healthy men and women aged 50-70 years.

Subjects were first screened on the telephone. All women were post-menopausal. Hormone replacement was allowed; however, women subjects who were taking hormone replacement were asked to continue their hormone during the exercise training period. Those not on hormone replacement were asked not to take hormone replacement during the study.

In addition, to be eligible, all subjects were required to have a body mass index (BMI) less than 35 kg/m². Subjects were also excluded if they had orthopedic problems that would or could interfere with the exercise training. Subjects were also excluded if they had known cardiovascular diseases, uncontrolled hypertension (BP>160/90 mmHg), diabetes, liver diseases, renal diseases, or hematological diseases.

All subjects were non-smokers and were not taking any medication known to affect blood coagulation or fibrinolysis at the time of testing. Screening for blood chemistry ensured no overt diabetes, liver diseases, renal diseases, or hematological diseases. Subjects were screened with a medical history and general physical examination. To screen for occult cardiovascular disease, graded exercise testing with 12-lead electrocardiogram (ECG) was administered under medical supervision.

### Example 2

### Testing Procedures

After completing the Dietary Stabilization phase of the study, subjects underwent baseline tests that included blood samples for fibrinolysis enzymes: PAI-1 and t-PA activities, and t-PA antigen. Other baseline tests included blood samples for lipid profiles, body weight, body composition, and VO₂max. All tests were repeated at the end of the study.

### Example 3

### Blood Sample Collections

Subjects were not allowed to take anti-inflammatory drugs or other medications known to affect fibrinolysis for 24 hours before blood sample collections. Recent infection/inflammation can cause an acute rise in PAI-1 and other acute phase proteins, which may influence the levels of fibrinolysis enzymes. Thus, a questionnaire regarding recent (less than 1 month) infection/inflammation was administered before performing phlebotomies. The information from the questionnaire was used to help interpret the fibrinolysis assays. For instance, a person who had recent infection/inflammation did not have a sample drawn until he or she did not suffer from inflammation or infection for two weeks. Blood samples were drawn after a 12-hour over-night fast. To avoid the effect of circadian variations on the fibrinolytic enzymes 12 blood samples were drawn between 6:30-9:30 am. Upon arrival at the phlebotomy laboratory, subjects were asked to rest quietly for 20 minutes in a sturdy armed chair. After the rest period, phlebotomy was performed without stasis in most cases or minimal stasis in some cases. All blood samples were collected from an antecubital vein by a 21G ¾-butterfly needle with 7-inch long tubing connected with luer adapter.

To determine PAI-1 activity, 4.5 ml of blood was drawn directly into a standard size vacuum tube containing 0.5 ml of 0.5 M citrate buffer at pH 4.5 (Biopool Stabilyte, Biopool, Sweden) to acidify the blood sample. This acidified condition is critical to stop further reaction between t-PA and PAI-1, and to prevent the release of inactive PAI-1 during in vitro platelet degranulation.

To determine t-PA antigen activity, 4.5 ml of blood was directly collected into a vacuum tube containing 3.8% trisodium citrate (0.129 M) with the final volume ratio of whole blood and anticoagulant of 9:1. Blood samples were collected only if the venous blood flowed freely. The first 10 ml of blood was drawn into a vacuum tube containing EDTA anticoagulant for blood lipid profile determination. Immediately after each blood sample was collected, it was placed on ice and transferred to the centrifuge. Immediately (no later than 15 minutes) after phlebotomy, blood samples for fibrinolytic determinations were centrifuged at 10,000 rpm for 20 minutes at 4° C. After centrifugation, plasma was separated into 250-280 μl aliquots and stored at −80° C. until analysis. All blood samples for enzyme activity assays were measured within 6 months at the laboratory in VA Medical Center, Baltimore, Md.

Baseline and final citrated blood samples were analyzed for t-PA antigen levels in the same assay run at the end of the study to eliminate inter-assay variation. Inter-assay variability is established and well accepted in the Baltimore Va. Thrombosis and Haemostasis Laboratory for t-PA activities and t-PA antigen. Determination of inter-assay variability for fibrinolytic assays for the present study is mentioned in the sections below. Intra-assay variability was calculated from duplicate samples for all measurements conducted. To avoid a decline in enzymatic activities, only one freeze-thaw cycle was allowed.

### Example 4

### Fibrinolytic Enzyme Assays

Standardized procedures for blood fibrinolysis assays have been published previously (for example, in Meade T W, Ruddock V, Stirling Y, Chakrabarti R, Miller G J. Fibrinolytic activity, clotting factors, and long-term incidence of ischaemic heart disease in the Northwick Park Heart Study. Lancet 1993; 342:1076-1079; Hamsten A, de Faire U, Walldius G, Dahlen G, Szamosi A, Landou C, Blomback M, Wiman B. Plasminogen activator inhibitor in plasma: risk factor for recurrent myocardial infarction. Lancet 1987; 2:3-9; Cortellaro M, Cofrancesco E, Boschetti C, Mussoni L, Donati M B, Cardillo M, Catalano M, Gabrielli L, Lombardi B, Specchia G. Increased fibrin turnover and high PAI-1 activity as predictors of ischemic events in atherosclerotic patients. A case-control study. The PLAT Group. Arterioscler Thromb 1993; 13:1412-1417; Juhan-Vague I, Pyke S D, Alessi M C, Jespersen J, Haverkate F, Thompson S G. Fibrinolytic factors and the risk of myocardial infarction or sudden death in patients with angina pectoris. ECAT Study Group. European Concerted Action on Thrombosis and Disabilities. Circulation 1996; 94:2057-2063; Ridker P M, Vaughan D E, Stampfer M J, Manson J E, Hennekens C H. Endogenous tissue-type plasminogen activator and risk of myocardial infarction. Lancet 1993; 341:1165-1168; and Ridker P M, Hennekens C H, Stampfer M J, Manson J E, Vaughan D E. Prospective study of endogenous tissue plasminogen activator and risk of stroke. Lancet 1994; 343:940-943). The individual fibrinolytic enzyme analysis of the previous invention, however, was performed as specifically described below.

### PAI-1 Activity Assay

Platelet poor plasma PAI-1 activities were assayed by the chromogenic method using a commercial assay kit (Spectrolyse/pL PAI, Biopool, Sweden). The assay procedure was performed in microplates according to the instructional inserts, incorporated by reference in their entirety herein, provided with the assay kit and the Biopool Stabilyte tube package. The PAI-1 activity assay measures the inhibitory capacity of PAI-1 against t-PA. A two-step, indirect enzymatic assay was utilized to detect PAI-1 activity levels in plasma samples. This method has been described by Chmielewska et al. (U.S. Pat. No. 5,876,968), incorporated by reference in its entirety herein. In step one, an excessive, fixed amount of t-PA was added to the plasma samples to react with the existing PAI-1 in the samples. In each test tube, 25 μL of plasma sample was mixed with 50 μL of 20 IU/mL t-PA and incubated for 15 minutes. To destroy α₂-antiplasmin, 100 μL acetate buffer was added and the tube containing sample mixture was incubated in a water bath at 37° C. for 20 minutes.

In step two, the residual t-PA in the samples, after reacting with PAI-1, was indirectly measured from plasmin activity. To do so, 200 μL of a mixture of Glu-plasminogen, poly-D-lysine, and D-But-CHT-Lys-pNA at neutral pH was added to the 20 μL of samples in the microplate wells. All samples were tested in duplicate, whereas the standards were performed in triplicate according to the instructional insert. The Glu-plasminogen is a substrate for t-PA, poly-D-lysine is a stimulator for the reaction, and D-But-CHT-Lys-pNA is a chromogenic substrate for plasmin, which was converted from plasminogen by the residual t-PA. A dual wavelength mode (405 and 490 nm) was used to measure the t-PA activity in the microplate reader (Vmax Kinetic Microplate Reader, Molecular Devices).

The estimated amount of PAI-1 is inversely proportional to the amount of residual t-PA measured. One unit (AU) of PAI-1 activity is arbitrarily defined as the amount of PAI-1 that inhibits 1 international unit (IU) of human single-chain t-PA, which has been calibrated against the international standard for t-PA lot 86/670 distributed by NIBSAC (Holly Hill, London, England). The assay detection range was between 8-28 U/mL. The intra-assay and inter-assay coefficients of variation for PAI-1 activity were 5.8% and 8.6%. The intra-assay coefficient of variation was determined from all duplicates in the assays. The inter-assay coefficient of variation was determined from 15 different samples with a mean of approximately 12 AU/mL, analyzed on 2 different assay kits on the same day.

### t-PA Activity Assay

Platelet poor plasma t-PA activities were assayed by the direct amidolytic activity method using a commercial assay kit (Coaset t-PA, Chromogenix). The assay was mainly performed as directed by the manufacturer instructional insert, incorporated by reference in its entirety herein. A slight modification of the original instructional insert was used for the microplate assay. The principle of the measurement is based on the measurement of the amidolytic activity of plasmin, which is converted from plasminogen by t-PA present in the plasma. In each test tube, a plasma sample was diluted by adding 1.75 mL of water into 50 μL of plasma. Next, 100 μL diluted plasma samples were added into the microplate wells before adding 100 μL of a plasminogen/S2251/Tris buffer mixture. The t-PA converting activity of plasminogen to plasmin is substantially enhanced in the presence of fibrin(ogen), a t-PA stimulator. Therefore, 50 μL of the t-PA stimulator was added to the sample in each well and then the amidolytic activity of plasmin on the chromogenic substrate S-2251 was measured. A dual wavelength (405 and 490 nm) mode was used to determine the activity of plasmin on the color development due to a release of p-nitroaniline (pNA). The amidolytic activity of plasmin is linearly correlated within the range of 0.25-10 IU/mL t-PA activity in plasma. According to the manufacturer insert, this assay detects a lowest plasma t-PA activity of 0.10 IU/mL. The t-PA activities for this study were calibrated against the International Standard for t-PA from human melanoma cells (lot 83/517, National Institute for Biological Standards and Control, London, UK). The intra-assay coefficient of variation was calculated from all sample values obtained from all assay runs. The inter-assay variability for the laboratory has been determined and well accepted. The inter-assay coefficient of variation for the present study was determined from the values obtained from one sample analyzed in 2 different assay runs with a mean of 0.40 IU/mL. The intra-assay and inter-assay coefficients of variation were 5.5% and 1.8%, respectively.

### t-PA Antigen Assay

The quantitative analysis of t-PA antigen was performed from platelet poor plasma by enzyme-linked immunosorbent assay (ELISA), using a commercial assay kit (Asserachrom, Diagnotica Stago). The assay was performed according to the instructional insert provided by the manufacturer, said insert being incorporated by reference in its entirety. The principle for the method is based on the specificity of the 2 monoclonal antibodies to the t-PA antigen. The procedures were performed as follows. First, 200 μL of the 1:5 diluted plasma samples was added into the wells pre-coated with mouse monoclonal anti-human t-PA antibody. The antibody captures the t-PA present in the plasma samples within 2 hours of incubation. The wells were then washed with washing solution 5 times. Immediately after the washes, 200 μL of a second mouse monoclonal anti-human antibody specific to a different antigenic determinant of t-PA was added into each well. Another 2-hour incubation after adding the second antibody, which has been coupled with peroxidase, allows the t-PA and the 2 antibodies to form the “sandwich.” The second wash was performed 5 times to eliminate all unbound antibodies. The amount of peroxidase present in each well directly represents the amount of t-PA concentration in the plasma sample. The amount of peroxidase was revealed as its activity after adding 200 μL substrate ortho-phenylenediamine and hydrogen peroxide. The reaction was allowed to occur for 6 minutes. Subsequently, 100 μL HCl (1M) was added to stop the reaction. After the stop reagent was present for 10 minutes, the absorbance was measured at 490 nm with a microplate reader (Vmax Kinetic Microplate Reader, Molecular Devices). The external control provided by the manufacturer was included in each assay run to ensure the assay integrity. The intra-assay coefficient of variation, determined from all sample values, for this study was 3.9%. The inter-assay coefficient of variation of 2.8% was determined from the external control (value=38 ng/mL) provided by the assay kit manufacturer.

### Example 5

### Genotyping Technique

Deoxyribonucleic acid (DNA) samples were extracted from peripheral blood leukocytes using the salting out protocol. Genotype analysis for the PAI-1 promoter polymorphism was performed as mainly described by Margaglione et al., which is incorporated by reference in its entirety. The target DNA sequences were amplified by polymerase chain reaction (PCR) and the restriction fragment sizes were determined after an endonuclease restriction digest. The specific oligonucleotide primer set for PCR was obtained from the DNA Synthesis Facility, University of Pittsburgh, Pa. The forward and reverse primer sequences were a 22-mer (-697/-676) 5′-CACAGAGAGAGTCTGGCCACGT-3′ and a 21-mer (-598/-619) 5′ CCMCAGAGGACTCTTGGTCT-3′. The forward primer contained a base substitution at the position -681 (C-681A) to create a restriction site for the enzyme Bsl I in the 5G allele sequence. The enzyme recognition site is 5′-CCNNNNN\check{}NNGG-3′ 3′-GGNN\hat{ }NNNNNCC-5′.

The 4G allele would have “GGGG” inserted at the cut mark (as noted above with a carrot) and the 5G allele would have “GGGGG” inserted at the cut mark.

The PCR was performed on 50 μl of sample-reagent mixture, for 35 cycles in a thermocycler (PTC-225, Pertier Thermal Cycler). Each microplate well contained 1.5 μL of concentrated genomic DNA sample, 5.0 μ1 of 10× reaction buffer, 0.3 μM of each primer, 1.5 μL of 50 mM MgCl₂. 200 μM of dNTPs, and 1 U of thermostable Taq DNA polymerase (Gibco DRL, Life Technologies). An amount of 20 μL of the PCR products were digested by 5 units of restriction enzyme Bsl I (New England Biolabs Inc., Beverly, Mass., USA) at 55° C. overnight. The results of the endonuclease restriction digest were identified as 77 and 22 bp fragments of the 5G allele (containing 99 bp prior to digestion) and the 98 bp fragment of the non-cutting 40 allele. The restricted products were identified by 3.5% agarose gel electrophoresis in IX TBE buffer for 4 hours at 100 volts. The DNA fragments were stained with ethidium bromide and visualized under UV light. All genotype analyses were performed at the Department of Human Genetics at the University of Pittsburgh, Pittsburgh, Pa.

### Example 6

After subjects completed the screening, dietary stabilization, and baseline tests, they then were classified into 3 groups according to their PAI-1 genotype. Exercise training personnel were blinded to the genotype of the subjects to prevent preferential treatment. At the end of 6 months, subjects who completed the training program donated 2 blood samples, to assess fibrinolytic enzyme levels and lipoprotein variables, within 24-36 hours after their last bout of exercise. All other baseline tests were also repeated.

Table 1 sets for the mean fibrinolytic values obtained by the above examples.

TABLE 1
Baseline and final values of fibrinolytic measures among 3 PAI-1
genotype groups following moderate exercise training for six months

| | 4G/4G | 4G/5G | 5G/5G | |
|---|---|---|---|---|
| | (n = 15) | (n = 20) | (n = 10) | P ANOVA |
| PAI-1 activity, | | | | |
| IU/mL | | | | |
| Baseline | 16.4 ± 1.6 | 13.1 ± 0.9 | 15.4 ± 2.0 | 0.189 |
| Final | 15.3 ± 1.5 | 13.3 ± 1.1 | 15.0 ± 2.0 | 0.541 |
| Change | −1.1 ± 1.1 | 0.1 ± 1.3 | −0.5 ± 2.2 | 0.802 |
| t-PA activity, | | | | |
| U/mL | | | | |
| Baseline | 0.49 ± 0.08 | 0.60 ± 0.08 | 0.54 ± 0.06 | 0.574 |
| Final | 0.87 ± 0.14 | 1.09 ± 0.35 | 0.63 ± 0.14 | 0.556 |
| Change | 0.38 ± 0.11 | 0.50 ± 0.34 | 0.09 ± 0.15 | 0.611 |
| t-PA, antigen, | | | | |
| ng/mL | | | | |
| Baseline | 8.3 ± 1.0 | 7.6 ± 0.6 | 7.9 ± 0.9 | 0.795 |
| Final | 7.3 ± 0.9 | 6.7 ± 0.5 | 7.3 ± 0.7 | 0.736 |
| Change | −1.0 ± 0.3 | −0.9 ± 0.3 | −0.6 ± 0.4 | 0.721 |

TABLE 2
Baseline and final values of fibrinolytic measures among 3 t-PA
genotype groups following moderate exercise training for six months

| | II(n = 14) | ID(n = 24) | DD(n = 7) | P ANOVA |
|---|---|---|---|---|
| t-PA activ- | | | | |
| ity, U/mL | | | | |
| Baseline | 0.45 ± 0.05 | 0.62 ± 0.08 | 0.50 ± 0.06 | 0.228 |
| Final | 0.82 ± 0.13 | 1.1 ± 0.30 | 0.45 ± 0.09 | 0.381 |
| Change | 0.38 ± 0.10 | 0.48 ± 0.28 | −0.05 ± 0.07 | 0.510 |
| t-PA, anti- | | | | |
| gen, ng/mL | | | | |
| Baseline | 8.2 ± 0.9 | 7.8 ± 0.6 | 7.3 ± 1.1 | 0.827 |
| Final | 7.4 ± 0.8 | 6.7 ± 0.5 | 7.5 ± 0.7 | 0.622 |
| Change | −0.8 ± 0.3 | −1.2 ± 0.2 | 0.17 ± 0.7 | 0.054 |

The results set forth in Table 1 show that PAI-1 levels in subjects change with moderate exercise training. Further, the data shows that there is a tendency for subjects with a 4G/4G genotype to respond better than a subject with a 4G/5G or a 5G/5G genotype. The data of Table 1 also shows that t-PA activity also changes in relation with the introduction of moderate exercise. The results describe a tendency for subjects with a 4G/4G genotype to respond better to exercise treatment that individuals with a 4G/5G or 5G/5G genotype. Table 1 also shows that t-PA antigen levels are also adjusted by exercise and that there is a tendency for subjects with a 4G/4G genotype to respond better to exercise treatment than a subject with a 4G/5G genotype. Therefore, the data shows that 4G homozygotes respond the best to exercise, with 5G homozygotes either responding less beneficially or not at all.

Table 2 contains data that also shows that 4G carriers respond the best in terms of t-PA activity and antigen levels to exercise training.

These results show that endurance exercise training, whether that training is extensive, moderate, or limited, increases fibrinolysis in middle-aged to older men and women by increasing the t-PA activity, and, thereby decreasing t-PA antigen levels. Further, it has been determined that individuals with at least one 4G allele responds more favorably to endurance exercise training than an individual without a 4G allele and that an individual with two 4G alleles responds even more favorably than a person with either one 4G allele or no 4G alleles.

We claim:

1. A method of increasing fibrinolysis levels in a subject, the method comprising:
identifying a subject with at least one 4G allele and/or genotype at the plasminogen activator inhibitor-1 (PAI-1) gene promoter site; and
engaging the subject in exercise training for a period of time sufficient to increase fibrinolysis in the subject.

2. The method of claim 1, wherein the subject has a 4G/5G genotype.

3. The method of claim 1, wherein the subject has a 4G/4G genotype.

4. The method of claim 1, wherein the exercise training comprises extensive exercise.

5. The method of claim 1, wherein the exercise training comprises moderate exercise.

6. The method of claim 1, wherein the exercise training comprises limited exercise.

4. A method of preventing cardiovascular disease in a subject, the method comprising:
identifying a subject with at least one 4G allele and/or genotype at the plasminogen activator inhibitor-1 (PAI-1) gene,promoter site; and
engaging the subject in exercise training for a period of time sufficient to prevent cardiovascular disease in the subject.

5. The method of claim 4, wherein the subject has a 4G/5G genotype.

6. The method of claim 4, wherein the subject has a 4G/4G genotype.

7. The method of claim 4, wherein the exercise training comprises extensive exercise.

8. The method of claim 4, wherein the exercise training comprises moderate exercise.

9. The method of claim 4, wherein the exercise training comprises limited exercise.

10. A method of ameliorating cardiovascular disease in a subject suffering from cardiovascular disease, the method comprising:
identifying a subject with at least one 4G allele and/or genotype at the plasminogen activator inhibitor-1 (PAI-1) gene promoter site; and
engaging the subject in exercise training for a period of time sufficient to ameliorate cardiovascular disease in the subject.

11. The method of claim 10, wherein the subject has a 4G/5G genotype.

12. The method of claim 10, wherein the subject has a 4G/4G genotype.

13. The method of claim 10, wherein the exercise training comprises extensive exercise.

14. The method of claim 10, wherein the exercise training comprises moderate exercise.

15. The method of claim 10, wherein the exercise training comprises limited exercise.
